(19) United States
(12) Patent Application Publication
(10) Pub. No.: US 2007/0099871 A1
(43) Pub. Date: May 3, 2007

(54) Bioreductively-activated prodrugs

(75) Inventors: Peter David Davis, Oxford (GB); Matthew Alexander Naylor, Middlesex (GB); Peter Thomson, Middlesex (GB); Steven Albert Everett, Middlesex (GB); Michael Richard Lacey Stratford, Middlesex (GB); Peter Wardman, Middlesex (GB)

Correspondence Address: HAMILTON, BROOK, SMITH & REYNOLDS, P.C., 530 VIRGINIA ROAD, P.O. BOX 9133, CONCORD, MA, 01742-9133, US

(73) Assignees: ANGIOGENE PHARMACEUTICALS LIMITED, Oxfordshire (GB); GRAY LABORATORY CANCER RESEARCH TRUST, Middlesex (GB)

(21) Appl. No.: 10/550,864

(22) PCT Filed: Mar. 26, 2004
(86) PCT No.: PCT/GB04/01330

§ 371 Date: Oct. 27, 2005

(30) Foreign Application Priority Data
Mar. 26, 2003 (GB) 0306907.7

(51) Int. Cl. A61K 31/675; A61K 31/404; A61K 31/122; A61K 31/137; A61K 31/66; C07F 9/572; C07F 9/28; C07D 403/02

(52) U.S. Cl. 514/80; 514/418; 514/414; 514/682; 548/415; 548/512; 548/465; 552/294; 552/296

(57) ABSTRACT

The present invention relates to a compound of formula (1), or a pharmaceutically acceptable salt thereof, wherein: Ar is a substituted aryl or heteroaryl group bearing at least one nitro or azido group or is a group of formula (2) or (3) wherein R₁, and R₂, which may be the same or different are independently optionally substituted alkyl, optionally substituted alkenyl, optionally substituted alkynyl, aryl, COR₃ or, together with the intervening carbon atom, form an optionally substituted heterocycloalkyl or carbocyclic ring; L is —OC(O)— or —OP(O)(OR₆)—; n is 0 or 1; X is 0, S, NR₇ or a single covalent bond; R₃ is OR₄ or NR₄R₅; R₄, R₅, R₆ and R₇ are each independently hydrogen or optionally substituted alkyl or, where Rᵣ is NR₄R₅, R₄ and R₅ can be joined to form, together with the intervening nitrogen atom, a heterocycloalkyl ring; R₈ is hydrogen, alkoxy or dialkylaminoalkyl; R₉ is optionally substituted alkyl; Rio is hydrogen, alkyl, alkoxy or dialkylaminoalkyl; R₁₁ and R₁₂ are independently hydrogen, alkyl, alkoxy, thioalkoxy, amino, alkylamino, dialkylamino, morpholino, piperidino, piperazino or 1=aziridinyl; A is an optionally substituted aryl or heteroaryl ring; and Dr is a moiety such that DrXH represents a cytotoxic or cytostatic compound.

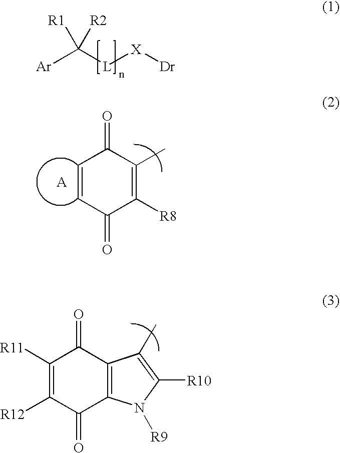

This invention relates to compounds useful in the treatment of cell proliferation disorders. More particularly the invention relates to a series of compounds that are activated under hypoxic conditions.

Many drugs used in conventional cancer chemotherapy are toxic to growing cancer cells but lack complete specificity. Thus other normal tissues are affected and ensuing side effects limit the dose that can be administered. Therefore the exposure of the cancerous tumour to the compound, and in turn the effectiveness of the therapy, is limited. Recent research has shown promising clinical activity of compounds, such as protein kinase inhibitors, which are cytostatic in their action. However the specificity of such compounds is not complete and side effects arising from action against normal tissues can again limit the effectiveness of therapy. There is a need for drugs that target the tumour more selectively.

Many solid tumours exhibit regions of hypoxia (low oxygen tension). Inadequate blood supply to the central regions of the tumour results in hypoxia that can be chronic or acute. This hypoxia represents a challenge to effective therapy by radiation or by conventional chemotherapy since hypoxic regions are often more resistant to these modalities. It has been suggested, however, that tumour hypoxia can be used to target tumours for drug action (Kennedy, Cancer Res. 1980, 40, 2356-2360). One particular method of using the hypoxic regions of tumours for drug targeting is the selective activation of prodrugs under conditions of low oxygen tension. A concept has been advanced whereby the activity of a cytotoxic compound can be masked by a trigger moiety which, under hypoxic conditions, mediates fragmentation of the masked cytotoxic compound into the active cytotoxic agent (Denny, Lancet Oncol 2000, 1, 25-9). Compounds attempting to utilize this concept typically consist of the trigger moiety attached, often via a linker moiety, to a cytotoxic moiety (the effector).

Hypoxia is also a feature of the rheumatoid arthritic joint (Rothschild Semin Arthritis Rheum 1982, 12, 11-31). Cell proliferation is also a feature of the arthritic joint. Systemic antiproliferative drugs (for example methotrexate) are used in the therapy of rheumatoid arthritis but are limited by side effects. Psoriatic lesions are also characterized by cell proliferation and hypoxia (Dvorak Int Arch Allergy Immunol. 1995, 107, 233-5). Hypoxia drives proliferation of endothelial cells in the retina in diabetic retinopathy and in the choroid of the eye in wet age-related macular degeneration (Das, Prog Retin Eye Res 2003, 22, 721-48). In addition to the well-documented hypoxia of solid tumours, sites where leukemic cells are proliferating, for example bone marrow and spleen, can also be hypoxic (Jensen, Cell Prolif 2000, 33, 381-95).

A number of hypoxic trigger moieties have been disclosed including nitrobenzenes, nitronaphthalenes, nitroimidazoles, nitrofurans, nitrothiophenes, nitropyrroles, nitropyrazoles, benzoquinones, naphthoquinones, indoloquinones and azidobenzenes (for some examples see Naylor, Mini Rev. Med. Chem: 2001 1, 17-29; Tercel, J. Med. Chem. 2001, 44, 3511-3522 and Damen, Bioorg. Med. Chem. 2002, 10, 71-77).

A number of effector moieties have been utilised in the art including nitrogen mustards, phosphoramide mustards, taxanes, enediynes and indole derivatives (for some examples see Naylor, loc cit and Papot, Curr. Med. Chem. Anti Cancer Agents 2002, 2, 155-185).

Hypoxic triggers joined to effectors via a linking group have been described wherein the linking group consists of a carbonate or carbamate (for some examples see Naylor, and Papot loc cit). In these cases it is intended that the intermediate carbonic acid or carbamic acid, formed by the initial hypoxia-driven fragmentation, further fragments to give the active agent.

Despite a body of work regarding compounds that break down selectively under low oxygen tensions to release an anticancer agent, no such compound is yet in clinical use. A number of problems have been encountered in the development of such compounds. A lack of stability of the prodrugs towards non-bioreductive processes has been regularly encountered. For example Sartorelli (J Med Chem 1986, 29, 84-89) has described a series of 5-fluorouracil prodrugs designed to fragment to give 5-fluorouracil under hypoxic conditions but these compounds did not prove useful in this respect due to chemical instability. Borch (J Med Chem 2000, 43, 3157-3167) has described a series of naphthoquinones designed to release phosphoramide mustards on quinone reduction but these compounds were unstable in cell cytotoxicity assays and released the active agent by mechanisms other than quinone reduction. Similarly the carbonate-linked taxol prodrugs described by Damen (loc cit) were reported to be unstable towards enzymatic hydrolysis in cellular assays, thereby releasing taxol by a non-reductive process. Borch (J Med Chem 2001, 44, 74-77) has also described a series of hypoxia activated nitroheterocyclic phosphoramidates which were unstable in vivo, displaying rapid metabolism and consequent elimination half-lives of only a few minutes. Wilson (J Med Chem 2001, 44, 3511-3522) has disclosed a series of nitroheteroaryl quaternary salts as bioreductive prodrugs of mechlorethamine but concluded that the compounds were too unstable with regard to non-specific release of mechlorethamine to be of use as bioreductive agents. Thus prodrugs showing improved stability towards non-reductive processes would have advantage.

A further consideration is the rate of release of the active drug under hypoxic conditions. To be effective the bioreductively activated prodrug needs to deliver the drug at a rate which competes with clearance of the prodrug and diffusion of the drug out of the solid tumour. Prodrugs that fragment faster than those in the art, or that fragment more efficiently at oxygen tensions commonly found in solid tumours, would be advantageous.

It is an object of this invention to provide prodrugs that on bioreductive activation break down to release a cytotoxic or cytostatic agent.

Thus according to one aspect of the invention we provide a compound of formula (1) or a pharmaceutically acceptable salt thereof: 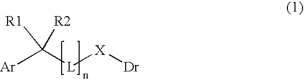
wherein:
Ar is a substituted aryl or heteroaryl group bearing at least one nitro or azido group or is a group of formula (2) or (3) 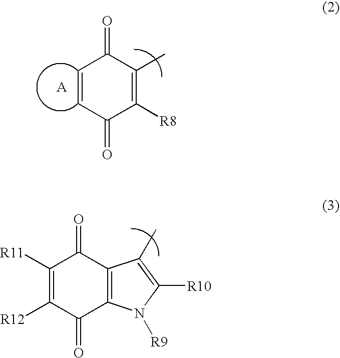
R₁ and R₂, which may be the same or different are independently optionally substituted alkyl, optionally substituted alkenyl, optionally substituted alkynyl, aryl, COR₃ or, together with the intervening carbon atom, form an optionally substituted heterocycloalkyl or carbocyclic ring;
L is —OC(O)—, or —OP(O)(OR₆)—;
n is 0 or 1;
X is O, S, NR₇ or a single covalent bond;
R₃ is OR₄ or NR₄R₅;
R₄, R₅, R₆ and R₇ are each independently hydrogen or optionally substituted alkyl or, where R₃ is NR₄R₅, R₄ and R₅ can be joined to form, together with the intervening nitrogen atom, a heterocycloalkyl ring;
R₈ is hydrogen, alkoxy or dialkylaminoalkyl;
R₉ is optionally substituted alkyl;
R₁₀ is hydrogen, alkyl, alkoxy or dialkylaminoalkyl;
R₁₁ and R₁₂ are independently hydrogen, alkyl, alkoxy, thioalkoxy, amino, alkylamino, dialkylamino, morpholino, piperidino, piperazino or 1-aziridinyl;
A is an optionally substituted aryl or heteroaryl ring; and
Dr is a moiety such that DrXH represents a cytotoxic or cytostatic compound. Examples of compounds of formula (1) include those wherein:
Ar is a substituted aryl or heteroaryl group bearing at least one nitro or azido group or is a group of formula (2) or (3), as defined above;
R₁ and R₂, which may be the same or different are independently optionally substituted alkyl, optionally substituted alkenyl, optionally substituted alkynyl, aryl, COR₃ or, together with the intervening carbon atom, form an optionally substituted heterocycloalkyl or carbocyclic ring;
L is —OC(O)— or —OP(O)(OR₆)—,
n is 0 or 1;
X is O, S, NR₇ or a single covalent bond;
R₃ is OR₄ or NR₄R₅;
R₄, R₅, R₆ and R₇ are each independently hydrogen or alkyl;
R₈ is hydrogen, alkoxy or dialkylaminoalkyl;
R₉ is optionally substituted alkyl;
R₁₀ is hydrogen, alkoxy or dialkylaminoalkyl;
R₁₁ and R₁₂ are independently hydrogen, alkyl, alkoxy, thioalkoxy, amino, alkylamino, dialkylamino or 1-aziridinyl;
A is an optionally substituted aryl or heteroaryl ring; and
Dr is a moiety such that DrXH represents a cytotoxic or cytostatic compound.

As used herein the term “alkyl”, alone or in combinations, means a straight or branched-chain alkyl group containing from one to seven, preferably a maximum of four, carbon atoms such as methyl, ethyl, propyl, isopropyl, butyl, sec-butyl, t-butyl and pentyl. Typically, alkyl group or moiety is a linear or branched alkyl group or moiety containing from 1 to 6 carbon atoms, such as a C₁-C₄ or C₁-C₂ alkyl group or moiety.

As used herein, alkoxy is a said alkyl group which is attached to an oxygen atom.

As used herein, a thioalkoxy group is a said alkyl group which is attached to a a sulphur atom.

An alkenyl group may be for example an olefinic group containing from two to seven carbon atoms, for example ethenyl, n-propenyl, i-propenyl, n-butyenyl, i-butenyl, s-butenyl and t-butenyl. Typically an alkenyl group is a C₂-C₆ alkenyl group, for example a C₂-C₄ alkenyl group. An alkenyl group typically contains only one double bond.

As used herein, an alkyl group is a linear or branched alkynyl group, typically an alkynyl group is a C₂-C₆, for example a C₂-C₄ alkynyl group, for example ethynyl, n-propynyl or n-butynyl. Typically, an alkynyl group contains only one triple bond. An alkynyl group may be for example an ethynyl, propynyl or butynyl group.

Optional substituents which may be present on alkyl, alkenyl or alkynyl groups include one or more substituents selected from halogen, amino, monoalkylamino, dialkylamino, hydroxy, alkoxy, alkylthio, alkylsulphonyl, aryl, heteroaryl, acylamino, alkoxycarbonylamino, alkanoyl, acyloxy, carboxy, sulphate or phosphate groups. A further example of an optional substituent which may be present on alkyl, alkenyl or alkynyl groups is a heterocycloalkyl group. Preferably, the substituents on an alkyl, alkenyl or alkynyl group are selected from halogen, amino, mono(C₁-C₄ alkyl)amino, di(C₁-C₄ alkyl)amino, hydroxy, C₁-C₄ alkoxy, C₁-C₄ alkylthio or (C₁-C₄ alkyl)sulphonyl groups. Typically, alkyl, alkenyl or alkynyl groups are unsubstituted or substituted by one, two or three substituents. Typically, said substituents which may be present on alkyl, alkenyl or alkynyl groups are themselves unsubstituted. More preferably, an alkyl, alkenyl or alkynyl group is unsubstituted or substituted by 1, 2 or 3 halogen atoms.

The term “halogen” means fluorine, chlorine, bromine or iodine.

The term aryl means an unsubstituted phenyl group or a phenyl group carrying one or more, preferably one to three, substituents examples of which are halogen, optionally substituted alkyl, hydroxy, nitro, azido, cyano, ammo and alkoxy. Preferably, an aryl group is an unsubstituted phenyl group or a phenyl group substituted with 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy. More preferably, an aryl group is a phenyl group which is unsubstituted or substituted with 1, 2, or 3 unsubstituted substituents selected from halogen, C₁-C₂ alkyl, C₁-C₂ haloalkyl, C₁-C₂ alkoxy and C₁-C₂ haloalkoxy substituents.

As used herein, a haloalkyl or haloalkoxy group is a said alkyl or alkoxy group, substituted by one or more said halogen atoms. Typically, a haloalkyl or haloalkoxy group is substituted by 1, 2 or 3 said halogen atoms. Preferred haloalkyl and haloalkoxy groups include perhaloalkyl and perhaloalkoxy groups such as —CY₃ and —OCY₃ wherein Y is said halogen atom, for example chlorine or fluorine. Particularly preferred haloalkyl groups are —CF₃ and —CCl₃. Particularly preferred haloalkoxy groups are —CF₃ and —CCl₃.

The term heteroaryl is defined herein as a monocyclic or bicyclic aromatic group containing one to four heteroatoms selected in any combination from N, S or O atoms. Typically, the bicyclic aromatic group is a fused bicyclic aromatic group. A heteroaryl group is typically a 5- to 10-membered ring, such as a 5- or 6-membered ring, containing at least one heteroatom, for example 1, 2, or 3 heteroatoms chosen from N, S or O atoms. Examples of heteroaryl groups include pyridyl, pyrimidyl, furyl, thienyl, pyrrolyl pyrazolyl, indolyl, benzofuryl, benzothienyl, benzothiazolyl, oxazolyl, isoxazolyl, thiazolyl, isothiazolyl, imidazolyl, triazolyl, quinolyl and isoquinolyl groups. A heteroaryl group can carry one or more, preferably one to three, substituents examples of which are halogen, optionally substituted alkyl, hydroxy, nitro, azido, cyano, amino and alkoxy. Preferably, a heteroaryl group is an unsubstituted heteroaryl group or a heteroaryl group substituted with 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy. More preferably, a heteroaryl group is unsubstituted or substituted with 1, 2, or 3 unsubstituted substituents selected from halogen, C₁-C₂ alkyl, C₁-C₂ haloalkyl, C₁-C₂ alkoxy and C₁-C₂ haloalkoxy substituents.

A heterocycloalkyl ring is typically a non-aromatic, saturated or unsaturated C₃₋₁₀ carbocyclic ring in which one or more, for example, 1, 2 or 3, of the carbon atoms are replaced by a heteroatom selected from N, O or S. Saturated heterocycloalkyl groups are preferred. The term heterocycloalkyl ring includes heterocycloalkyl groups containing 3-6 carbon atoms and one or two oxygen, sulphur or nitrogen atoms. Particular examples of such groups include azetidinyl, pyrrolidinyl, piperidinyl, homopiperidinyl, piperazinyl, homopiperazinyl, morpholinyl or thiomorpholinyl groups.

Substituents which may be present on a heterocycloalkyl ring include one or more groups selected from optionally substituted alkyl, halogen, oxo, hydroxy, alkoxy, alkylthio, amino, alkylamino, dialkylamino, carboxy, alkoxycarbonyl, aminocarbonyl, alkylaminocarbonyl, dialkylaminocarbonyl, alkylsulphonyl, aminosulphonyl, acylamino, alkoxycarbonylamino, alkanoyl, acyloxy, sulphate, phosphate and alkylphosphate. Preferably, a heterocycloalkyl ring is an unsubstituted heterocycloalkyl group or a heterocycloalkyl group substituted with 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy. More preferably, a heterocycloalkyl ring is unsubstituted or substituted with 1, 2, or 3 unsubstituted substituents selected from halogen, C₁-C₂ alkyl, C₁-C₂ haloalkyl, C₁-C₂ alkoxy and C₁-C₂ haloalkoxy substituents.

The term carbocyclic ring means a cycloaliphatic group containing 3-10 carbon atoms such as, for example, cyclopropyl, cyclobutyl, cyclopentyl or cyclohexyl. The cycloaliphatic group is saturated or unsaturated. Typically, the cycloaliphatic ring is saturated. Typically a carbocylic group contains from 3 to 8, for example from 3 to 6 carbon atoms. Substituents which may be present on a carbocyclic ring include one or more groups selected from optionally substituted alkyl, halogen, oxo, hydroxy, alkoxy, alkylthio, amino, alkylamino, dialkylamino, carboxy, alkoxycarbonyl, aminocarbonyl, alkylaminocarbonyl, dialkylaminocarbonyl, alkylsulphonyl, aminosulphonyl, acylamino, alkoxycarbonylamino, alkanoyl, acyloxy, sulphate, phosphate and alkylphosphate. Preferably, a carbocyclic group is an unsubstituted heteroaryl group or a heteroaryl group substituted with 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy. More preferably, a carbocyclic group is unsubstituted or substituted with 1, 2, or 3 unsubstituted substituents selected from halogen, (C₁-C₂)alkyl, (C₁-C₂)haloalkyl, C₁-C₂ alkoxy and C₁-C₂ haloalkoxy substituents.

Cytostatic or cytotoxic compounds represented by DrXH are known or can be determined by standard methods known to those skilled in the art. Such methods include in vitro assays of cell growth using cancer cell lines. Examples of such methods include DNA synthesis assays such as thymidine incorporation assays, protein stain assays such as sulphorhodamine B assays, vital stain assays such as neutral red assays, dye reduction assays such as MTT assays and dye exclusion assays such as trypan blue assays. Appropriate cytotoxic or cytostatic compounds represented by DrXH inhibit cell growth by at least 50% in one or more in vitro assays. Thus one skilled in the art can determine the group Dr in formula (1).

Typically, the activity of such a cytotoxic or cytostatic compound can be assessed by:
(a) seeding A549 cells in Eagles Minimum Essential Medium supplemented with 10% foetal calf serum and non-essential amino acids at 10³ cell per well on a 96 well plate;
(b) incubating for 24 hours to allow the cells to attach,
(c) exposing the cells to test compound dissolved in DMSO and diluted with cell culture medium for 6 hours and incubating for a further 72 hours; and
(d) assessing the number of viable cells in each well.

Typically, step (d) is conducted by adding MTS tetrazolium compound (Owen's reagent) to each well and leaving for 4 hours and then measuring the absorbance at 490 nm with a 96 well plate reader.

Typically, a said cytotoxic or cytostatic compound shows activity in the above assay at a concentration below 1 mM. More typically, it shows activity at a concentration below 250 nM.

More useful values of the groups Dr and X in formula (1) are those for which the compound DrXH is active in one or more in vitro assays of cell growth at concentrations below 1 mM.

Most useful values of the groups Dr and X in formula (1) are those for which the compound DrXH is more potent as a cytotoxic or cytostatic agent, as determined by standard methods, than the corresponding compound of formula (1).

The moiety Dr may be attached to X such that the group XH in DrXH represents a phenolic or alcoholic hydroxyl group, a carboxylic acid OH group, a thiol group, an anilino group, an alkylanilino group, an amino group or an alkylamino group.

Where n is 0 and X is a single covalent bond, the bond represented by X will typically be attached to a heterocyclic nitrogen atom in the drug moiety Dr.

Non-limiting examples of DrXH include compounds selected from an anthracyclin antibiotic such as doxorubicin and daunorubicin; an antimetabolite such as 5-fluorouracil, 6-mercaptopurine, 6-thioguanine, cytarabine, gemcitabine, capecitabine, fludarabine, cladribine, trimetrexate and methotrexate; a topoisomerase inhibitor such as an epipodophyllotoxin derivative for example etoposide and teniposide or such as a camptothecin derivative, for example topotecan and SN38; and an inhibitor of mitosis for example a combretastatin derivative such as combretastatin A4, combretastatin A1, and podophyllotoxin, a vinca alkaloid such as vinblastine, vincristine and vinorelbine, a taxane derivative such as paclitaxel and docetaxel, an epothilone derivative such as epothilone B, epothilone D, deoxyepothilone B and BMS 247550, a dolastatin derivative and a cryptophycin derivative. Non-limiting examples of DrH also include inhibitors of protein kinases such as, for example, the anilinoquinazoline inhibitors of protein tyrosine kinases for example gefitinib, erlotinib, ZD6474 and AZD2171. Further non-limiting examples of DrH include antagonists of (6R)-5,6,7,8-tetrahydrobiopterin. A further example of a suitable anthracyclin antibiotic is epirubibin. Further examples of suitable antimetabolites include decitabine (5-aza-2′-deoxycytidine), troxacitabine (2′-deoxy-3′-oxacytidine), 5-azacytidine, 4′-thioaracytidine, tezacitabine and clofasabine.

Where DrXH represents 6-mercatopurine, 6-thioguanine or an analogue thereof and n is 0 the group Ar—CR₁R₂ in compounds of formula (1) can conveniently be attached at the S(6) position of the drugs so as to form thioether prodrugs.

Where DrXH represents a cytosine analogue such as cytarabine, gemcitabine, capecitabine, decitabine (5-aza-2′-deoxycytidine), troxacitabine (2′-deoxy-3′-oxacytidine), 5-azacytidine, 4′-thioaracytidine or tezacitabine the group Ar—CR₁R₂C-(L)ₙ can conveniently be attached at the N⁴-position of the drugs.

Where DrXH represents an adenosine analogue such as fludarabine, clofarabine or cladribine, the group Ar—CR₁R₂-(L)ₙ can conveniently be attached at the N⁶-position of the drugs.

Where DrXH represents a combretastatin analogue such as combretastatin A4 or combretastatin A1, the group Ar—CR₁R₂-(L)ₙ can conveniently be attached via a phenolic oxygen in the combretastatin B-ring.

Where DrXH represents an epipodophyllotoxin derivative for example etoposide and teniposide the group Ar—CR₁R₂-(L)ₙ can conveniently be attached at the 4′ position of a 4′demethylepipodophyllotoxin as a phenolic ether.

Where DrXH represents a camptothecin analogue or a homocamptothecin analogue the group Ar—CR₁R₂-(L)ₙ can conveniently be attached at a phenolic oxygen or nitrogen at the 10-position of the camptothecin.

Where DrXH represents a taxane analogue the group Ar—CR₁R₂-(L)ₙ can conveniently be attached via the 2′ hydroxy group.

When R₁ and R₂, together with the carbon to which they are attached, form a heterocycloalkyl or carbocyclic ring, said ring is typically a 3 to 10 membered heterocycloalkyl ring or a C₃₋₁₀ carbocyclic ring, which ring is unsubstituted or substituted by 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy.

More typically, when R₁ and R₂, together with the carbon to which they are attached, form a heterocycloalkyl or carbocyclic ring, said ring is typically a 5 to 6 membered heterocycloalkyl ring or a C₅₋₆ carbocyclic ring, which ring is unsubstituted or substituted by 1, 2 or 3 unsubstituted substituents selected from halogen, C₁-C₂ alkyl, C₁-C₂ haloalkyl, C₁-C₂ alkoxy and C₁-C₂ haloalkoxy.

Preferably, when R₁ and R₂, together with the carbon to which they are attached, form a heterocycloalkyl or carbocyclic ring, said ring is a 5 to 6 membered heterocycloalkyl ring, for example a piperidyl ring, which ring is unsubstituted or substituted by one unsubstituted C₁-C₂ alkyl group.

Typically, when R₁ and R₂, together with the carbon to which they are attached, do not form a heterocycloalkyl or carbocyclic ring, R₁ and R₂ are the same or different and each represent unsubstituted C₁-C₆ alkyl, unsubstituted C₁-C₆ alkenyl, unsubstituted C₁-C₆ alkynyl, a COR₃ group or a phenyl group which is unsubstituted or substituted with 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy.

More typically, when R₁ and R₂, together with the carbon to which they are attached, do not form a heterocycloalkyl or carbocyclic ring, R₁ and R₂ are the same or different and each represent unsubstituted C₁-C₄ alkyl, unsubstituted C₁-C₄ alkenyl, unsubstituted C₁-C₄ alkynyl, a COR₃ group or a phenyl group which is unsubstituted or substituted with 1, 2 or 3 unsubstituted substituents selected from halogen, C₁-C₄ alkyl, hydroxy, amino, C₁-C₂ haloalkyl, C₁-C₂ alkoxy and C₁-C₂ haloalkoxy.

Typically, R₃ is hydroxy, unsubstituted C₁-C₄ alkoxy or NR₄R₅, wherein R₄ and R₅ are the same or different and each represent hydroxy or unsubstituted C₁-C₄ alkoxy, or R₄ and R₅ form, together with the nitrogen atom to which they are attached, a 3 to 10 membered heterocycloalkyl ring, which ring is unsubstituted or substituted by 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy.

Preferably, R₃ is hydroxy, unsubstituted C₁-C₂ alkoxy or NR₄R₅, wherein R₄ and R₅ are the same or different and each represent hydrogen or unsubstituted C₁-C₂ alkyl.

Most preferably, when R₁ and R₂, together with the carbon to which they are attached, do not form a heterocycloalkyl or carbocyclic ring, R₁ and R₂ are the same or different and each represent unsubstituted C₁-C₂ alkyl or an unsubstituted —CO₂—(C₁-C₂ alkyl)- group.

Typically, in the compound of formula (1), L is —OC(O)— or —OP(O)(OR₆)—, wherein R₆ is hydrogen or unsubstituted C₁₋₆ alkyl. Preferably R₆ is hydrogen or unsubstituted C₁₋₄ alkyl. Preferably, L is —OC(O)—.

Typically, in the compound of formula (1), X is O, S, a single covalent bond or NR₇, wherein R₇ is hydrogen or unsubstituted C₁-C₆ alkyl, for example unsubstituted C₁-C₄ alkyl. Preferred examples of X are O, S and NH. One particularly useful group of compounds of formula (1) are those in which n is 0 and X is O or S. Another useful group of compounds of formula (1) are those in which n is 1 and X is NH.

Typically, R₈ is hydrogen, unsubstituted C₁-C₄ alkoxy or unsubstituted di(C₁-C₆ alkyl)amino(C₁-C₆ alkyl). More typically, R₈ is hydrogen or unsubstituted C₁-C₂ alkoxy.

Typically, R₉ is unsubstituted C₁-C₆ alkyl, for example unsubstituted C₁-C₄ alkyl.

Typically, R₁₀ is hydrogen, unsubstituted C₁₋₆ alkyl, unsubstituted C₁₋₄ alkoxy or unsubstituted di(C₁-C₆ alkyl)amino(C₁-C₆alkyl). More typically, R₁₀ is hydrogen, unsubstituted C₁-C₄ alkyl or unsubstituted C₁-C₂ alkoxy.

Typically, R₁₁ and R₁₂ are each unsubstituted substituents selected from hydrogen, C₁₋₆ alkyl, C₁₋₄ alkoxy, thio(C₁-C₄)alkoxy, amino, (C₁-C₆)alkylamino, di(C₁-C₆)alkylamino, morpholino, piperidino, piperazino and 1-aziridinyl substituents. More typically, R₁ and R₁₂ are each selected from hydrogen, unsubstituted C₁₋₄ alkyl and unsubstituted C₁₋₂ alkoxy.

A is typically a phenyl group or a 5 or 6 membered heteroaryl ring. Typically, the phenyl group or heteroaryl ring is unsubstituted or substituted with 1, 2 or 3 unsubstituted substituents selected from halogen, C₁-C₄ alkyl, hydroxy, amino, C₁-C₄ haloalkyl, C₁-C₄ alkoxy and C₁-C₄ haloalkoxy substituents. Preferably, the phenyl group or heteroaryl ring is unsubstituted or substituted with 1 or 2 unsubstituted substituents selected from halogen, C₁-C₂ alkyl and C₁-C₂ haloalkyl.

Typically, in the compound of formula (1), Ar is a substituted aryl or 5- to 10-membered heteroaryl group bearing at least one nitro or azido group. Preferably, Ar carries one substituent selected from a nitro or azido group and 0, 1 or 2 further unsubstituted substituents chosen from halogen, C₁-C₆ alkyl, hydroxy, amino, C₁-C₄ haloalkyl, C₁-C₄ alkoxy and C₁-C₄ haloalkoxy substituents. Preferably, said further substituents are chosen from halogen, unsubstituted C₁-C₄ alkyl, hydroxy and amino substituents. Typically, when Ar is a substituted aryl or 5- to 10-membered heteroaryl group bearing at least one nitro or azido group, it is a phenyl or 5- to 6-membered heteroaryl group carrying one substituent selected from a nitro or azido group and 0, 1 or 2 said further substituents. More preferably, when Ar is a substituted aryl or 5- to 10-membered heteroaryl group bearing at least one nitro or azido substituent, said group carries only one substituent which substituent is chosen from a nitro or azido group. Preferably, said substituent is a nitro group.

Typically, Ar is phenyl or a 5- or 6-membered heteroaryl group, for example an imidazolyl or thienyl, substituted by only one substituent which substituent is a nitro group. Preferred values of Ar include unsubstituted groups selected from nitrophenyl; nitroimidazole, nitrothiophene and nitrofuranyl groups. A particularly useful group of compounds of formula (1) are those in which Ar is a 5-nitrothien-2-yl group, a 5-nitrofuran-2-yl group or a 1-methyl-2-nitroimidazol-5-yl group. Preferred examples of Ar include 4-nitrophenyl, 1-methyl-2-nitroimidazolyl-5-yl and 5-nitrothien-2-yl.

Preferably, in the compound of formula (1), Dr is a moiety such that DrXH is combretastatin A4, etoposide, cytarabine or 6-mercaptopurine.

Preferably, in the compound of formula (1),
either (a) R₁ and R₂, together with the carbon atom to which they are attached, form a 3 to 10 membered heterocycloalkyl ring or a C₃₋₁₀ carbocyclic ring, which ring is unsubstituted or substituted by 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy, or (b) R₁ and R₂ are the same or different and each represent unsubstituted C₁-C₆ alkyl, unsubstituted C₁-C₆ alkenyl, unsubstituted C₁-C₆ alkyl, a COR₃ group, a phenyl group which is unsubstituted or substituted with 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy;
R₃ is hydroxy, unsubstituted C₁-C₄ alkoxy or NR₅, wherein R₄ and R₅ are the same or different and each represent hydroxy or unsubstituted C₁-C₄ alkoxy, or R₄ and R₅ form, together with the nitrogen atom to which they are attached, a 3 to 10 membered heterocycloalkyl ring, which ring is unsubstituted or substituted by 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy;
n is 0 or 1, wherein when n is 1, L is —OC(O)— or —OP(O)(OR₆)—;
R₆ is hydrogen or unsubstituted C₁₋₆ alkyl;
X is O, S, a single covalent bond or NR₇;
R₇ is hydrogen or unsubstituted C₁₋₆ alkyl;

Ar is a substituted aryl or 5 to 10 membered heteroaryl group which carries one substituent selected from a nitro or azido group and 0, 1 or 2 further unsubstituted substituents chosen from halogen, C₁-C₆ alkyl, hydroxy, amino, C₁-C₄ haloalkyl, C₁-C₄ alkoxy and C₁-C₄haloalkoxy substituents; and

Dr is a moiety such that DrXH is an anthracyclin antibiotic, an antimetabolite, a topoisomerase inhibitor, an inhibitor of mitosis, inhibitors of protein kinases or an antagonists of (6R)-5,6,7,8-tetrahydrobiopterin.

More preferably, in the compound of formula (1),

either (a) when R₁ and R₂ together with the carbon to which they are attached form a 5 to 6 membered heterocycloalkyl ring, which ring is unsubstituted or substituted by one unsubstituted C₁-C₂ alkyl group; or (b) R₁ and R₂ are the same or different and each represent unsubstituted C₁-C₂ alkyl or an unsubstituted —CO₂—(C₁-C₂ alkyl) group;
n is 0 or 1, wherein when n is 1, L is —OC(O)—;
X is O, S or NH;
Ar is 4-nitrophenyl, 1-methyl-2-nitroimidazolyl-5-yl or 5-nitrothien-2-yl; and
Dr is a moiety such that DrXH is combretastatin A4, etoposide, cytarabine or 6-mercaptopurine.

Most preferably, the compound of formula (1) is selected from 1-(4-Methoxy-3-(2-(5-nitrothiophen-2-yl)propan-2-yl)oxyphenyl-2-(3,4,5-trimethoxy)phenyl-Z-ethene, 1-(4-Methoxy-3-(2-(4-nitrophenyl)propan-2-yl)oxyphenyl-2-(3,4,5-trimethoxy)phenyl-Z-ethene, 9-(7,8-Dihydroxy-2-methyl-hexahydro-pyrano[3,2-d][1,3]-dioxin-6-yloxy)-5-{3,5-dimethoxy-4-[1-methyl-1-(4-nitrophenyl)-ethoxy]-phenyl}-5,8,8a,9-tetrahydro-5aH-furo[3′,4′:6,7]naphtho[2,3-d][1,3]dioxol-6-one, 6-(2-(4-nitrophenyl)propan-2-ylsulfanyl)-9H-purine, 1-(4-Methoxy-3-(1-methyl-4-(5-nitrothien-2-yl)piperidin-4-yl)oxycarbonyloxy)phenyl-2-(3,4,5-trimethoxy)phenyl-Z-ethene, 1-(4-Methoxy-3-(2-(1-methyl-2-nitroimidazol-5-yl)propan-2-yl)oxyphenyl-2-(3,4,5-trimethoxy)phenyl-Z-ethene, 6-(2-(5-nitrothien-2-yl)propan-2-ylsulfanyl)-9H-purine, N⁴-(2-(5-nitrothien-2-yl)prop-2-yl)oxycarbonyl-1-β-D-arabinofuranosylcytosine, 1-(3-(1-Ethoxycarbonyl-1-(5-nitrothien-2-yl)ethoxy)-4-methoxy-phenyl)-2-(3,4,5-trimethoxyphenyl)-Z-ethene and N-(2-{3-[1-Methyl-1-(5-nitro-thiophen-2-yl)-ethoxy]-phenyl}-ethyl)-acetamide.

Where one or more functional groups in compounds of formula (1) are sufficiently basic or acidic the formation of salts is possible. Suitable salts include pharmaceutically acceptable salts for example acid addition salts including hydrochlorides, hydrobromides, phosphates, sulphates, hydrogen sulphates, alkylsulphonates, arylsulphonates, acetates, benzoates, citrates, maleates, fumarates, succinates, lactates and tartrates, salts derived from inorganic bases including alkali metal salts such as sodium or potassium salts, alkaline earth metal salts such as magnesium or calcium salts, and salts derived from organic amines such as morpholine, piperidine or dimethylamine salts.

Those skilled in the art will recognise that compounds of formula (1) may exist as stereoisomers and/or geometrical isomers and accordingly the present invention includes all such isomers which have anticancer activity and mixtures thereof.

A key and unifying feature of compounds of the present invention is the presence of the substituents R₁ and R₂. While not limiting on the invention it is believed that the presence of two substituents at this position confers advantage on the compounds by steric and/or electronic effects. For example the increased steric bulk provided by the two substituents can stabilize the compounds against release of the cytotoxic or cytostatic drug moiety by chemical or enzymatic processes other than the desired bioreductive processes. For another example the absence of a hydrogen atom alpha to the aromatic group prevents oxidation at this position; oxidation at this alpha position can lead to release of the effector outside of hypoxic regions. For another example the substituents R₁ and R₂ can extend the range of hypoxic oxygen tensions at which the cytotoxic or cytostatic moiety is released providing increased delivery of the cytotoxic or cytostatic compound to a solid tumour.

It is a further object of this invention to provide methods for the preparation of compounds of formula (1).

Compounds of formula (1) may be prepared by a number of processes as generally described below and more specifically in the Examples hereinafter. In the following process description, the symbols Ar, R₁, R₂, Dr, X, n, R₇ and R₈ when used in the formulae depicted are to be understood to represent those groups described above in relation to formula (1) unless otherwise indicated. In the schemes described below it may be necessary to employ protecting groups that are then removed during the final stages of the synthesis. The appropriate use of such protecting groups and processes for their removal will be readily apparent to those skilled in the art.

Compounds of formula (1) in which X is O or S and n is 0 can be prepared by Mitsunobu reaction of a tertiary alcohol of formula (4) with a phenol, thiophenol, carboxylic acid, thiocarboxylic acid, alcohol or thiol of formula (5) in a solvent such as an ether solvent, for example tetrahydrofuran, diethyl ether or dioxan or in a solvent such as an aromatic hydrocarbon for example benzene or toluene or in a solvent such as an aprotic solvent for example dimethylformamide, in the presence of a phosphine for example triphenylphosphine or tri-n-butylphosphine and in the presence of an azo compound such as diethylazodicarboxylate, diisopropylazodicarboxylate or 1,1′-(azodicarbonyl)dipiperidine at a temperature from about 0° C. to about the reflux temperature of the solvent, conveniently at room temperature.

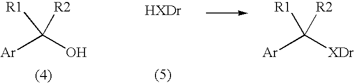

Alcohols of formula (4) are either known or can be prepared by standard methods apparent to one skilled in the art. Such methods include treatment of a ketone of formula (6) with an organometallic compound of formula (7) in which M represents a metal, metal halide or dialkylmetal, for example, Li, ZnBr, AlR₂, MgBr or MgI in a solvent such as an ether solvent, for example tetrahydrofuran or diethyl ether or in an aromatic solvent for example benzene or toluene at a temperature of between about −78° C. to about the reflux temperature of the solvent, preferably from about 0° C. to room temperature. Such methods also include the treatment of a ketone of formula (8) with an organometallic compound of formula (9) in which M represents a metal, metal halide or dialkylmetal, for example, Li, ZnBr, MgBr or MgI or dialkylaluminum in a solvent such as an ether solvent, for example tetrahydrofuran or diethyl ether or in an aromatic solvent for example benzene or toluene at a temperature of between about −78° C. to about the reflux temperature of the solvent, preferably from about 0° C. to room temperature. Where Ar is a substituted aryl or heteroaryl group bearing at least one nitro group such methods also include the aromatic electrophilic nitration of the appropriate aryl substrate with an appropriate nitrating agent at a temperature of between about −78° C. and room temperature. Appropriate nitrating agents are, for example, nitric acid in a solvent such as an acid anhydride for example acetic anhydride or in a solvent such as an acid for example sulphuric acid or acetic acid; nitronium tetrafluoroborate in a solvent such as an ether solvent, for example tetrahydrofuran or diethyl ether or in a solvent such as acetonitrile or glacial acetic acid or in a solvent such as a chlorinated solvent for example dichloromethane or dinitrogen tetroxide in a solvent such as an ether solvent, for example tetrahydrofuran or diethyl ether or in a solvent such as acetonitrile or glacial acetic acid or in a solvent such as a chlorinated solvent for example dichloromethane or in an aromatic solvent for example benzene or toluene.

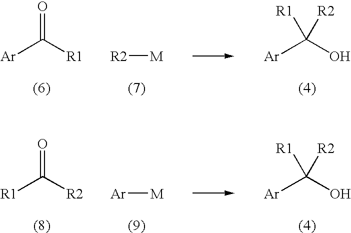

Compounds of formula (1) in which n=0 can also be prepared by treatment of a halide of formula (10), in which Hal represents a chlorine, bromine or iodine atom, with a compound of formula (5), in a solvent such as an aprotic solvent such as dimethylformamide or in an ether solvent such as diethyl ether or tetrahydrofuran, or in a ketone solvent such as acetone in the presence of a base such as a metal carbonate for example potassium carbonate or silver(I)carbonate or a base such as a metal hydride for example sodium hydride or potassium hydride, at a temperature of between about −78° C. to about the reflux temperature of the solvent preferably between 0° and room temperature.

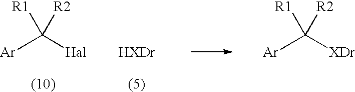

Halides of formula (10) are either known or can be prepared by standard methods apparent to one skilled in the art. Such methods include the halogenation of a compound of formula (11) with a halogenating agent such as N-bromosuccinimide, N-chlorosuccinimide or bromine in a solvent such as a chlorinated solvent for example dichloromethane or carbon tetrachloride at a temperature of about between about 0° C. and the reflux temperature of the solvent.

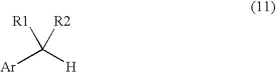

Compounds of formula (1) in which n is 0 and X represents an oxygen atom of a carboxyl group attached to Dr can be prepared by treatment of an alcohol of formula (4) with an acid chloride of formula DrC(O)Cl in a solvent such as a chlorinated solvent for example dichloromethane or trichloromethane at a temperature of between about 0° C. and the reflux temperature of the solvent conveniently in the presence of a base such as, for example, an amine base for example pyridine or triethylamine.

Compounds of formula (1) in which X is 0, n is 1 and L is —OC(O)— can be prepared by treatment of an alcohol of formula (4) with an acid chloride of formula DrOC(O)Cl in a solvent such as a chlorinated solvent for example dichloromethane or trichloromethane at a temperature of between about 0° C. and the reflux temperature of the solvent conveniently in the presence of a base such as, for example, an amine base for example pyridine or triethylamine.

Acid chlorides of formula DrOC(O)Cl are either known or can be prepared by standard methods apparent to one skilled in the art. Such methods include treatment of a compound of formula DrOH with phosgene or triphosgene in a solvent such as a chlorinated solvent for example dichloromethane or trichloromethane with or without the addition of dimethylformamide at a temperature of around 0° C. to room temperature.

Compounds of formula (1) in which X is NH, n is 1 and L is —OC(O)— can be prepared by treatment of an alcohol of formula (4) with an isocyanate of formula DrNCO in a solvent such as a chlorinated solvent for example dichloromethane or trichloromethane at a temperature of between about 0° C. and the reflux temperature of the solvent conveniently in the presence of a base such as, for example, an amine base for example pyridine or triethylamine.

Compounds of formula (1) in which X is NR₇, n is 1 and L is —OC(O)— can be prepared by treatment of a chloroformate of formula (12) with a compound of the formula DrNHR₇ in a solvent such as a chlorinated solvent for example dichloromethane or trichloromethane at a temperature of between about 0° C. and the reflux temperature of the solvent conveniently in the presence of a base such as, for example, an amine base for example pyridine or triethylamine.

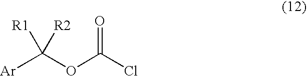

Compounds of formula (1) in which n is 1 and L is —OP(O)(OR₆)— can be prepared by treatment of an alcohol of formula (4) with a compound of the formula ClP(O)(OR₆)XDr in a solvent such as a chlorinated solvent for example dichloromethane or trichloromethane at a temperature of between about 0° C. and the reflux temperature of the solvent conveniently in the presence of a base such as, for example, an amine base for example pyridine or triethylamine.

Compounds of formula (1) in which n is 1, L is —OC(O)— and X is S can, of course, be made by the reaction of an appropriate acid chloride of formula Ar—CR₁R₂—O—C(O)Cl with a thiol, DrSH, in a solvent such as a chlorinated solvent for example dichloromethane or trichloromethane at a temperature of between about 0° C. and the reflux temperature of the solvent conveniently in the presence of a base such as, for example, an amine base for example pyridine or triethylamine.

Compounds of formula (1) can also be synthesized from other compounds of formula (1) by the application of standard methods, including substitution reactions, functional group transformations, bond-forming reactions and cyclisations known in the art.

The starting materials for the above described schemes are commercially available or can be synthesized using standard techniques.

Preparation of a compound of formula (1) as a single enantiomer or, where appropriate, diastereomer may be effected by synthesis from an enantiomerically pure starting material or intermediate or by resolution of the final product in a conventional manner.

The compounds of the invention may be administered as a sole therapy or in combination with other treatments. For the treatment of solid tumours compounds of the invention may be administered in combination with radiotherapy or in combination with other anti-tumour substances for example those selected from mitotic inhibitors, for example vinblastine, vincristine, vinorelbine, paclitaxel and docetaxel; alkylating agents, for example cisplatin, carboplatin, oxaliplatin, nitrogen mustard, melphalan, chlorambucil, busulphan and cyclophosphamide; antimetabolites, for example 5-fluorouracil, cytosine arabinoside, gemcitabine, capecitabine, methotrexate and hydroxyurea; intercalating agents for example adriamycin and bleomycin; enzymes, for example asparaginase; topoisomerase inhibitors for example etoposide, teniposide, topotecan and irinotecan; thymidylate synthase inhibitors for example raltitrexed; biological response modifiers for example interferon; antibodies for example edrecolomab, trastuzumab, bevacizumab and cetuximab; receptor tyrosine kinase inhibitors for example gefitinib, imatinib and erlotinib; and anti-hormones for example tamoxifen. Such combination treatment may involve simultaneous or sequential application of the individual components of the treatment.

For the prophylaxis and treatment of disease the compounds according to the invention may be administered as pharmaceutical compositions selected with regard to the intended route of administration and standard pharmaceutical practice. Such pharmaceutical compositions may take a form suitable for oral, buccal, nasal, topical, rectal or parenteral administration and may be prepared in a conventional manner using conventional excipients. For example for oral administration the pharmaceutical compositions may take the form of tablets or capsules. The compositions for oral administration may also be in the form of lozenges, aqueous or oily suspensions, dispersible powders or granules. For nasal administration or administration by inhalation the compounds may be conveniently delivered as a powder or in solution. Topical administration may be as an ointment or cream and rectal administration may be as a suppository. For parenteral injection (including intravenous, subcutaneous, intramuscular, intravascular or infusion) the composition may take the form of, for example, a sterile solution, suspension or emulsion. The compounds of the invention may also be administered as suppositories.

The dose of a compound of the invention required for the prophylaxis or treatment of a particular condition will vary depending on the compound chosen, the route of administration, the form and severity of the condition and whether the compound is to be administered alone or in combination with another drug. Thus the precise dose will be determined by the administering physician but in general daily dosages may be in the range 0.01 to 100 mg/kg preferably 0.1 to 10 mg/kg. Typically, daily dosage levels are from 0.05 mg to 2 g, for example from 5 mg to 1 g.

The present invention therefore provides a pharmaceutical composition comprising a compound of formula (1), or a pharmaceutically acceptable salt thereof, and a pharmaceutically acceptable carrier or diluent.

A further feature of the present invention is a compound of formula (1), or a pharmaceutically acceptable salt or solvate thereof, for use as a medicament. In particular, the present invention provides a compound of formula (1), or a pharmaceutically acceptable salt thereof, for the treatment of the human or animal body.

The compounds of the present invention are therapeutically useful in treating, preventing, ameliorating or reducing incidence of a proliferative disorder. Typically, the proliferative disorder is a hypoxic disorder. A hypoxic disorder is typically a disorder in which diseased cells are present in a hypoxic environment. Examples of the disorders that can be treated, prevented, ameliorated or disorders whose incidence can be reduced, include cancer, rheumatoid arthritis, psoriatic lesions, diabetic retinopathy or wet age-related macular degeneration.

Typically, the disorder is cancer. Preferably the cancer is a hypoxic cancer. A hypoxic cancer is, of course, a cancer wherein cancerous cells are in a hypoxic environment. Most preferably, the cancer is a solid tumour or leukaemia. Typically the leukaemia is leukaemia involving the spleen or bone marrow.

According to a further aspect of the invention there is provided the use of a compound of formula (1), or a pharmaceutically acceptable salt or solvate thereof, in the manufacture of a medicament for use in the therapy of a warm-blooded animal, for example a human, suffering from a proliferative disease for example cancer. In particular, the present invention provides the use of a compound of formula (1), or a pharmaceutically acceptable salt thereof, in the manufacture of a medicament for use in the treatment of the human or animal body, for the prevention or treatment of a said proliferative disorder.

According to a further aspect of the invention there is provided a compound of formula (1), or a pharmaceutically acceptable salt or solvate thereof, for use in a method of treatment of the human or animal body by therapy. In particular, the present invention provides a method of ameliorating or reducing the incidence of a said proliferative disorder in a patient, which method comprises administering to said patient an effective amount of a compound of formula (1), or a pharmaceutically acceptable salt thereof.

A number of enzymes are capable of reducing aryl and heteroaryl nitro groups. Strategies that increase the activity of such enzymes within solid tumours can therefore increase further the activity of prodrugs dependent on nitro reduction. Similarly a number of enzymes are capable of reducing quinones and indoloquinones and therefore similar strategies are possible to increase the effectiveness of drugs requiring activation by quinone reduction. Such strategies include linking such enzymes to a tumour-targeting antibody, administering such enzyme antibody conjugates to a host with a solid tumour then, after the conjugate has localised to the tumour, administering the prodrug. This approach is known as Antibody Directed Enzyme Prodrug Therapy (ADEPT). Alternatively the gene encoding for the enzyme might be delivered selectively and/or expressed selectively, in the tumour before administration of the prodrug. This approach is known as Gene Directed Enzyme Prodrug Therapy (GDEPT). When the gene is delivered by a viral vector the approach is sometimes known as Virus Directed Enzyme Prodrug Therapy (VDEPT).

Anlezark has disclosed nitroreductases and their use in an ADEPT strategy. Prodrugs for use in this strategy were also disclosed (US5633158 and US5977065). In WO 00 047725 Anlezark provides further disclosures of nitroreductase enzymes and their use in GDEPT strategies. Denny (WO 00 064864) has disclosed nitroaryl and nitroheteroaryl prodrugs for use in a GDEPT strategy. The use of quinone-reducing enzymes in ADEPT, GDEPT and MDEPT (Macromolecule Directed Enzyme Prodrug Therapy) is discussed in Skelly et al. Min Rev Med. Chem. 2001, 1, 293-306.

Thus it is a further object of this invention to provide the use of compounds of formula (1) in combination with a reductase, an antibody-reductase conjugate, a macromolecule-reductase conjugate or DNA encoding a reductase gene, in a method of treatment for the human body. Thus, the present invention provides a method of ameliorating or reducing the incidence of a said proliferative disorder in a patient, which method comprises administering to said patient an effective amount of
(a) a compound of formula (1), or a pharmaceutically acceptable salt thereof; and
(b) a reductase, an anti-body reductase conjugate, a macromolecule-reductase conjugate or DNA encoding a reductase gene.

Further, the present invention provides a product containing
(a) a, compound of formula (1), or a pharmaceutically acceptable salt thereof; and
(b) a reductase, an anti-body reductase conjugate, a macromolecule-reductase conjugate or DNA encoding a reductase gene
   for simulataneous, separate or sequential use in the treatment of a proliferative condition.

The ability of compounds of the invention to release cytotoxic or cytostatic agents selectively under hypoxic conditions can be assessed by using, for example, one or more of the procedures set out below:

Radiolysis

In the hypoxic environments of solid tumours, prodrugs can be reduced by one-electron processes that are inhibited in the normoxic environments of normal tissues. Radiolysis demonstrates the ability of bioreductively-activated prodrugs to release the active drug after one-electron reduction. Compounds were dissolved in an isopropanol/water mixture (50:50) at a concentration of 50 μM or below. Solutions, in gas-tight syringes, were saturated with nitrous oxide before irradiation in a ⁶⁰Co source at a dose rate of 3.9Gy min⁻¹ (as determined by Fricke dosimetry: H. Fricke and E. J. Hart, “Chemical Dosimetry” in Radiation Dosimetry Vol. 2 (F. H. Attrix and W. C. Roesch. Eds.), pp 167-239. Academic Press New York, 1966). Solutions were analysed for released drug by HPLC. The radiation chemical yields (G-values) obtained in this assay for selected example compounds are shown in Table 1.

TABLE 1
Radiation chemical yields from steady state radiolysis

| Compound of Example No. | Drug released | G (μmoles · J⁻¹) |
|---|---|---|
| 1 | Combretastatin A4 | 0.36 |
| 2 | Combretastatin A4 | 0.16 |
| 4 | 6-Mercaptopurine | 0.44 |
| 5 | Combretastatin A4 | 0.46 |
| 6 | Combretastatin A4 | 0.07 |
| 8 | Cytarabine | 0.38 |
| 9 | Combretastatin A4 | 0.50 |

Drug Release by Cytochrome p450 Reductase

Cytochrome p450 reductase is widely expressed in human tumours as well as in a range of normal tissues and is one of a number of enzymes that can catalyse bioreduction. This assay shows the ability of prodrugs to fragment into active drugs catalysed by cytochrome p450 selectively under conditions of low oxygen. Compounds were dissolved in DMSO to a concentration of 625 μM and 20 μL added to a mixture of 50 mmol dm⁻³ potassium phosphate buffer at pH 7.4 (2.4 mL), NADPH (20 μL of a 10 mM solution) and 60 μL of Supersomal™ p450 reductase (Gentest; Catalogue number P244) and incubated at 37° C. For experiments under nitrogen the mixture was degassed with nitrogen for 20 minutes prior to compound addition and overgassed with nitrogen during the incubation. Samples (100 μl) were taken at regular intervals and added to an equivalent volume of acetonitrile, then mixed and centrifuged at 14, 300 RPM for 2 min prior to product analysis by HPLC. In this test the compound of Example 1 produced combretastatin A4 at a rate of 710 ppmol·min⁻¹·mg protein⁻¹ under nitrogen but only 110 pmol·min⁻¹·mg protein⁻¹ under air.

Metabolism in Tumour Homogenates

Useful bioreductive prodrugs can be shown to release the active drug selectively under conditions of low oxygen in the presence of tumour homogenate in this assay. Freshly-excised CaNT tumours (approximately 0.5 to 1 g) were homogenised in 15 ml of ice-cold 50 mmol dm⁻³ potassium phosphate buffer at pH 7.4. The homogenates were centrifuged at 1000 RPM for 10 min and the supernatants stored on ice. The metabolism of 5 μmol dm⁻³ prodrug in air and N₂ was performed with 0.5 ml tumour homogenate (˜3 mg of protein by Bradford assay) with 100 mol dm⁻³ NADPH in 50 mmol dm⁻³ potassium phosphate buffer at pH 7.4 incubated at 37° C. Samples (60 μl) were taken at regular intervals and added to an equivalent volume of acetonitrile, then mixed and centrifuged at 14, 300 RPM for 2 min prior to product analysis by HPLC. In this test the compound of Example 1 produced combretastatin A4 at a rate of 120 pmol·min⁻¹·mg proteins⁻¹ under nitrogen but only 8 pmol·min⁻¹·mg protein⁻¹ under air.

Cellular Cytotoxicity

In a preferred embodiment of the invention the compounds of formula (1) will be less potent as cytotoxic or cytostatic agents than the corresponding cytotoxic or cytostatic compounds of formula DrXH which are released under hypoxic conditions. The cytotoxic or cytostatic properties of compounds of formula (1) and compounds of formula DrXH can be assessed for example, by use, for example, of this assay. The Celltiter 96® Aqᵤₑₒᵤₛ One Solution Cell Proliferation Assay kit (Promega Corporation, USA) which is a colorimetric method for determining the number of viable cells in proliferation or cytotoxicity assays was used. In this assay the MTS tetrazolium compound (Owen's Reagent) is bioreduced by viable cells into a coloured formazan product which is soluble in tissue culture medium and can be measured by recording absorbance at 490 nm with a 96 well plate reader. A549 cells were seeded in Eagles Minimum Essential Medium supplemented with 10% foetal calf serum and non-essential amino acids at 10 cell per well on a 96 well plate and allowed to attach for 24 h. Compounds were dissolved in DMSO and diluted with cell culture medium before addition. The cells were exposed to test compound (0 to 2 μmol dm⁻³) for 6 h then incubated for a further 72 h. The MTS reagent was added to each well, left for 4 h, then the absorbance measured at 490 nm with a 96 well plate reader. In this assay the compound of Example 1 had no activity at concentrations up to 2 M whereas combretastatin A4 reduced cell numbers to 50% of control at a concentration of around 250 nM.

Metabolism in Liver Homogenates

Metabolic stability of the compounds and unfavorable release of the drug by oxic liver can be assessed by using, for example, this assay. Freshly-excised mouse liver (approximately 1 g) was homogenised in 15 ml of ice-cold 50 mmol d-3 potassium phosphate buffer at pH 7.4. The homogenates were centrifuged at 1000 RPM for 10 min and the supernatants stored on ice. The metabolism of 5 mol dm⁻³ prodrug in air was performed with 0.5 ml liver homogenate (∫4 mg of protein by Bradford assay) with 100 μmol dm⁻³ NADPH in 50 mmol dm⁻³ potassium phosphate buffer at pH 7.4 incubated at 37° C. Samples (60 μl) were taken at regular intervals and added to an equivalent volume of acetonitrile, then mixed and centrifuged at 14, 300 RPM for 2 min prior to product analysis by HPLC. In this test the compound of Example 1 produced combretastatin A4 at a rate of only 3 pmol·min⁻¹·mg protein⁻¹. In contrast the corresponding compound 1-(4-methoxy-3-(5-nitrothiene-2-yl)methoxy)phenyl-2-(3,4,5-trimethoxy)phenyl-Z-ethene, lacking the key features of the invention produced combretastatin at a greater rate of 20 pmol·min⁻¹·mg proteins⁻¹.

The invention is illustrated by the following non-limiting Examples in which, unless otherwise stated:

DMF means dimethylformamide

THF means tetrahydrofuran

MeOH means methyl alcohol

EtOAc means ethyl acetate

DCM means dichloromethane

TLC means thin-layer chromatography

MeCN means acetonitrile

TFA means trifluoroacetic acid

LC-RT means the retention time given by high-performance liquid chromatography performed using a Waters Integrity system with detection by mass spectroscopy with electron impact ionization. Chromatography used a Hichrom RPB column (100×3.2 mm) with various solvent gradients of either A: 10% acetonitrile, water or B: 5% Acetonitrile, 0.1% TFA with C: Acetonitrile, at a flow rate of 0.5 ml/min.

### EXAMPLE 1

### 1-(4-Methoxy-3-(2-(5-nitrothiophen-2-yl)propan-2-yl)oxyphenyl-2-(3,4,5-trimethoxy)phenyl-Z-ethene

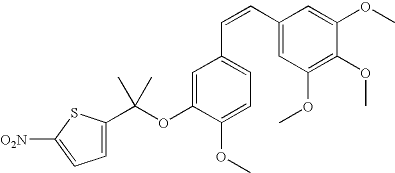

1-Methyl-1-(5-nitrothiophen-2-yl)ethanol (200 mg, 1.07 mmol) was dissolved in benzene (2.5 ml) together with combretastatin A4 (320 mg, 1 mmol) and 1,1-(azodicarbonyl)dipiperidine (ADDP, 250 mg, 1 mmol) and the solution maintained under argon with stirring. Tributylphosphine (200 mg, 1 mmol, dissolved in benzene (0.5 ml)) was then added via syringe and under argon. The solution was stirred for 24 h at 20° C. and then partitioned with EtOAc/water (100 ml) and the organic layer washed with brine (50 ml), dried (MgSO₄) and evaporated. The residue was purified by flash chromatography on silica gel (33% EtOAc/hexane) and then on a second silica column (DCM) to give a pale yellow oil (150 mg, 31%). ¹H NMR (500 MHz, CDCl₃) δ 7.78 (d, J=5 Hz, 1H), 7.05 (d, J=5 Hz, 1H), 6.86 (d, J=5 Hz, 1H), 6.81 (s, 1H), 6.74 (s, 1H), 6.475 (d, J=5 Hz, 4H), 3.89 (s, 3H), 3.85 (s, 3H), 3.76 (s, 3H), 3.75 (s, 3H), 1.63 (s, 3H), 1.60 (s, 3H) ppm. MS (m/z, %) 485 (M+, 4.3%), 316 (100%), 301-(56%). LC-RT 4.34 minutes (100% MeCN).

### EXAMPLE 2

### 1-(4-Methoxy-3-(2-(4-nitrophenyl)propan-2-yl)oxyphenyl-2-(3,4,5-trimethoxy)phenyl-Z-ethene

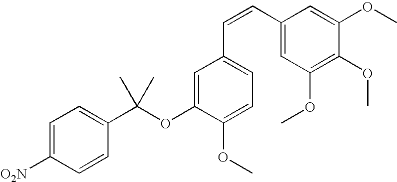

Sodium hydride (9 mg, 0.22 mmol) was added to combretastatin A4 (60 mg, 0.19 mmol) in DMF (0.2 mL). To this was added 2-bromo-2-(4-nitro)phenylpropane (54 mg, 0.22 mmol) in DMF (0.2 mL) and the reaction was stirred for 72 h. The reaction mixture was partitioned (EtOAc and brine), the aqueous phase was extracted (EtOAc), the organic phases were combined then dried (MgSO₄) and evaporated. Preparative TLC, using 10% EtOAc/hexane as solvent, yielded the product as a wax (8 mg, 9%); TLC R_{f}=0.15, 10% EtOAc/hexane; LC-RT 4.14 minutes (100% MeCN). MS (m/z, %) 479 M, 15%), 316 (100%), 301 (66%), 163 (15%), 149 (9%), 133 (40%). ¹H NMR (250 MHz, CDCl₃) δ 7.88 (2H, s, ArH), 7.33 (1H, s, ArH), 7.04 (1H, dd, J=8.3, 1.9, ArH), 6.87 (2H, m, 2×ArH), 6.41 (2H, s, CH═CH, 2×ArH), 6.33 (3H, m, CH═CH, ArH), 3.95 (3H, s, OCH₃), 3.89 (3H, s, OCH₃), 3.76 (6H, s, 2×OCH₃), 1.71 (6H, s, 2×CH₃) ppm.

### EXAMPLE 3

### 9-(7,8-Dihydroxy-2-methyl-hexahydro-pyrano[3,2-d][1,3]-dioxin-6-yloxy)-5-{3,5-dimethoxy-4-[1-methyl-1-(4-nitrophenyl)-ethoxy]-phenyl}-5,8,8a,9-tetrahydro-5aH-furo[3′,4′:6,7]naphtho[2,3-d][1,3]dioxol-6-one

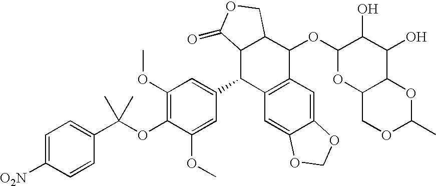

Sodium hydride (40 mg, 0.84 mmol) was added to a mixture of etoposide (144 mg, 0.56 mmol), 2-bromo-2-(4-nitro)phenylpropane (204 mg, 0.84 mmol) in DMF (0.5 mL) and the reaction was stirred for 72 h. The reaction mixture was partitioned (EtOAc and brine), the aqueous phase was extracted (EtOAc), the organic phases were combined then dried (MgSO₄) and evaporated. Preparative TLC, using. EtOAc as solvent, and then preparative HPLC afforded the product as a wax (8 mg, 2%); TLC R_{f}=0.7, EtOAc. LC-RT 6.29 minutes (TFA 50-100%). MS (m/z, %) 663 (1%), 401 (1%), 398 (1%), 382 (5%), 353 (1%), 324 (3%), 163 (100%), 150 (20%), 133 (80%). ¹H NMR (250 MHz, CDCl₃) δ 8.26 (2H, d, J=7.0, ArH), 7.91 (2H, d, J=7.0, ArH), 6.84 (1H, s, ArH), 6.56 (1H, s, ArH), 6.47 (1H, s, ArH), 6.41 (1H, s, ArH), 6.03 (1H, d, J=1.3, OCH₂O), 6.02 (1H, d, J=1.3, OCH₂O), 5.00 (1H, d, J=3.0, OCHO), 4.79 (1H, q, J=4.8, OCHO), 4.59 (2H, m, ArCHAr, ArCHCH), 4.27 (1H, d, J=4.8, OCH), 4.22 (1H, dd, J=4.8, OCH), 3.97 (1H, d, J=7.6), 3.71 (6H, s, OCH₃), 3.63 (2H, t, J=10.2, CO₂CH), 3.54 (1H, t, J=7.9, CHOH), 3.39 (1H, t, J=9.3, CHOH), 3.22 (2H, m, OCH, ArCHCH), 3.02 (1H, m, CH), 2.81 (1H, bs, OH), 2.69 (1H, bs, OH), 1.72 (3H, s, CH₃), 1.70 (3H, s, CH₃), 1.42 (3H, d, J=5.0, CH₃) ppm.

### EXAMPLE 4

### 6-(2-(4-nitrophenyl)propan-2-ylsulfanyl)-9H-purine

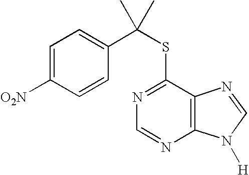

Sodium hydride (80 mg, 1.96 mmol) was added to 6-mercaptopurine (308 mg, 1.88 mmol) in DMF (2 mL). To this was added 2-bromo-2-(4-nitro)phenylpropane (400 mg, 0.98 mmol) in DMF (2 mL) and the reaction was stirred for 24 h. The reaction mixture was partitioned (EtOAc and brine), the aqueous phase was extracted (EtOAc), the organic phases were combined then washed (water then brine), dried (MgSO₄) and evaporated. Flash chromatography, eluting with 50% and 75% EtOAc/hexane then 100% EtOAc, afforded a fluffy white solid (101 mg, 33%); TLC R_{f}=0.48, EtOAc; mp 206-208° C.; LC-RT 4.2 minutes (TFA 50-100%). MS (m/z, %) 315 (M+, 8%), 163 (40%), 152 (100%), 133 (25%), 125 (20%). ¹H NMR (250 MHz, CDCl₃) δ 8.52 (1H, S, N═CR), 8.27 (1H, s, N═CH), 8.18 (2H, d, J=7.0, ArH), 7.92 (1H, d, J=7.0 ArH), 2.16 (6H, s, 2×CH₃) ppm.

### EXAMPLE 5

### 1-(4-Methoxy-3-(1-methyl-4-(5-nitrothien-2-yl)piperidin-4-yl)oxycarbonyloxy)phenyl-2-(3,4,5-trimethoxy)phenyl-Z-ethene

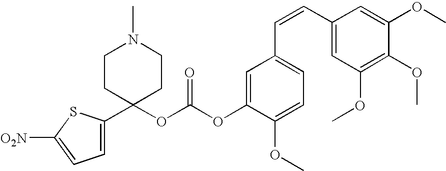

Phosgene (0.1 mL, 0.20 mmol, 20% solution in toluene) was added to DCM (0.5 mL) at 0° C. To this was added combretastatin A4 (56 mg, 0.18 mmol) in DCM (0.5 mL), followed after 1 hour by triethylamine (28 μL, 0.20 mmol). After 6 hours, the reaction mixture was added drop wise to a cooled (0° C.) solution of 4-hydroxy-1-methyl-4-(5-nitrothien-2-yl)piperidine (44 mg, 0.18 mmol), pyridine (15 μL, 0.18 mmol), DCM (1 mL) and DMF (1 mL). The reaction mixture was allowed to reach ambient temperature and stirred for a further 2 hours. The brown solution was partitioned (EtOAc, brine), aqueous phase extracted (EtOAc), organic phase washed (H₂O, brine), dried (MgSO₄) and concentrated in vacuo. Flash chromatography, eluting with 50% EtOAc/hexane, 100% EtOAc then 50% MeOH/EtOAc, furnished the desired product as an orange-brown wax (39 mg; 37%). R_{f}=0.34 (50% MeOH/EtOAc); ¹H NMR (500 MHz, CDCl₃) δ 7.87 (d, 1H, J=5.0 Hz, Ar—H), 7.18 (d, 1H, J=5.0 Hz, Ar—H), 7.12 (s, 1H, Ar—H), 7.10 (s, 1H, J=5.0 Hz, Ar—H), 6.88 (d, 1H, J=5.0 Hz, CH), 6.53 (s, 2H, Ar—H), 6.50 (d, 2H, J=5.0 Hz, Ar—H, CH), 3.87 (s, 3H, O—CH₃), 3.82 (s, 3H, O—CH₃), 3.74 (s, 6H, O—CH₃), 2.82 (bd, 2H, J=15.0 Hz, CH₂), 2.68 (bd, 2H, J=15.0 Hz, CH₂), 2.52 (bt, 2H, J=10.0 Hz, CH₂), 2.42 (s, 2H, N—CH₃), 2.25 (bt, 2H, J=10.0 Hz, CH₂) ppm; LC-R_{T} 5.14 minutes (TFA 50-100%); MS (m/z, %) 584 (M⁺, 1%), 316 (33%), 301 (40%), 225 (100%).

The 4-hydroxy-1-methyl-4-(5-nitrothien-2-yl)-piperidine used as starting material in the above preparation was prepared as follows: n-Butyllithium (14 mL, 22.4 mmol) was added to a solution of N,N-diisopropylamine (2.26 g, 22.4 mmol) in THF (80 mL) at −78° C. After 5 minutes, a solution of 2-nitrothiophene (2.47 g, 19.18 mmol) in THF (10 mL) was added drop-wise. After a further 5 minutes, a solution of 1-methyl-piperidin-4-one (2.53 g, 22.4 mmol) in THF (10 mL) was added and the reaction mixture stirred for a further 1 hour. The reaction was quenched with saturated NH₄Cl_{(aq)} and concentrated hydrochloric acid (2 mL) then allowed to reach ambient temperature. The reaction mixture was partitioned (EtOAc, H₂O), aqueous phase extracted (EtOAc), neutralised (saturated NaHCO_{3(aq)}) then re-extracted (EtOAc). The organic phase was then washed (H₂O, brine), dried (MgSO₄) and concentrated in vacuo to a brown oil. Flash chromatography, eluting with EtOAc, 50% MeOH/EtOAc and then 100% MeOH, afforded the desired product as a creamy brown solid (572 mg, 12%), mp 156-157° C.; ¹H NMR (60 MHz, CDCl₃) δ 7.81 (d, 1H, J=4.2 Hz, Ar—H), 6.91 (d, 1H, J=4.2 Hz, Ar—H), 2.68 (s, 3H, N—CH₃), 2.33-1.94 (m, 8H, CH₂) ppm; LC-RT 2.97 minutes (TFA 20-50%); MS (m/z, %) 242 (, 100%), 224 (50%), 197 (29%).

### EXAMPLE 6

### 1-(4-Methoxy-3-(2-(1-methyl-2-nitroimidazol-5-yl)propan-2-yl)oxyphenyl-2-(3,4,5-trimethoxy)phenyl-Z-ethene

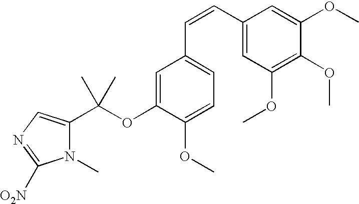

5-(1-Hydroxy-1-methylethyl)-1-methyl-2-nitro-1H-imidazole (10 mg, 0.054 mmol) was dissolved in THF (1.5 mL) together with triphenylphosphine (42 mg, 0.16 mmol) and combretastatin A4 (51 mg, 0.16 mmol). Diethylazodicarboxylate (28 mg, 0.16 mmol) was then added and the solution stirred for 18 h at room temperature. A further amount of 5-(1-hydroxy-1-methylethyl)-1-methyl-2-nitro-1H-imidazole (10 mg, 0.054 mmol) was then added and after a further 18 h the solution was applied directly to a silica column and eluted with 25% EtOAc/hexane to give the title compound as a yellow gum (30 mg, 15%). LC-RT 6.55 minutes (TFA 50-100%); MS (m/z, %) 484 (M⁺, 6%), 438 (6%), 317 (100%), 302 (54%), 170 (16%). ¹H NMR (250 MHz, CDCl₃) δ 7.32 (1H, s, HarH), 7.03 (1H, dd, J=8.5, 2.0, ArH), 6.86 (2H, t, J=4.8, ArH), 6.49 (4H, m, CH═CH, 2×ArH), 3.91 (3H, s, OCH₃), 3.81 (3H, s, OCH₃), 3.77 (6H, s, 2×OCH₃), 3.70 (3H, s, NCH₃), 1.68 (6H, s, 2×CH₃) ppm.

### EXAMPLE 7

### 6-(2-(5-nitrothien-2-yl)propan-2-ylsulfanyl)-9H-purine

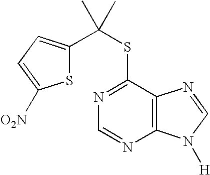

Sodium hydride (16 mg, 0.40 mmol) was added to 6-mercaptopurine hydrate (34 mg, 0.20 mmol) in DMF (1 mL) and the reaction was stirred for 2 h. The reaction mixture was added by Pasteur pipette to a solution of 2-chloro-2-(5-nitrothien-2-yl)propane and DMF (1 mL). After 2 h, the mixture was partitioned (ethyl acetate and brine), the aqueous phase was extracted (ethyl acetate), the organic phases were combined, washed (water then brine) then adsorbed on to flash silica in vacuo. Flash chromatography, eluting with DCM then 2% methanol/DCM, afforded a yellow oil (25 mg, 40%); TLC R_{f}=0.3, 10% methanol/DCM. ¹H NMR (500 MHz, CDCl₃) δ 8.62 (1H, s, N═CH), 8.16 (1H, s, N═CH), 7.78 (1H, d, J=5.0, ArH), 7.17 (1H, d, J=5.0, ArH), 2.19 (6H, s, 2×CH₃) ppm.

### EXAMPLE 8

### N⁴-(2-(5-nitrothien-2-yl)prop-2-yl)oxycarbonyl-1-β-D-arabiuofuranosylcytosine

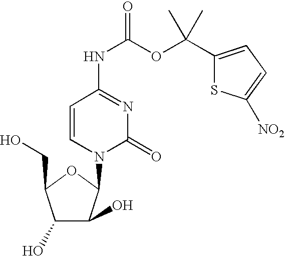

Potassium carbonate (2 mg, 0.01 mmol) was added to N⁴-(2-(5-nitrothien-2-yl)prop-2-yl)oxycarbonyl-1-β-D-triacetoxyarabinofuranosylcytosine (50 mg, 0.05 mmol) in THF (0.13 mL) and methanol (0.13 mL). The reaction was stirred for 12 h then filtered through a flash silica pad; the pad was washed with methanol, and the filtrate evaporated. Flash chromatography, eluting with ethyl acetate then consecutively with 2%, 5%, 15%; and 20% methanol/ethyl acetate, afforded the title compound as a waxy white solid (16 mg, 67%); TLC R_{f}=0.5, 10% methanol/ethyl acetate; mpt 127-129° C. ¹H NMR (500 MHz, d6-DMSO) δ 10.78 (1H, s, —NH), 8.04 (1H, d, J=5.0, HarH), 8.00 (1H, d, J=10, NCH), 7.26 (1H, d, J=5.0, HarH), 6.87 (1H, d, J=10.0, NCH═CH), 6.04 (1H, d, J=5.0, NCHO), 5.45 (2H, s, J=5.0, 2×OH), 5.02 (1H, t, J=5.0, OH), 4.06 (1H, bs, CHOH), 3.92 (1H, bs, OCHCH₂OH), 3.92 (1H, bs, CHOH), 3.61 (2H, m, J=5.0, CH₂OH), 1.88 (6H, s, 2×CH₃) ppm. The N⁴-(2-(5-nitrothien-2-yl)prop-2-yl)oxycarbonyl-1-β-D-triacetoxyarabinofuranosylcytosine used in the above preparation was prepared as follows:

2-(5-nitrothien-2-yl)propan-2-ol (152 mg, 0.81 mmol), triacetyl-Ara-C (300 mg, 0.74 mmol), pyridine (126 uL, 1.55 mmol) and DCM (2 mL) were stirred at 0° C. A solution of phosgene (0.8 mL, 1.48 mmol, 2M in toluene) was added dropwise to the reaction mixture and stirring continued for 72 h. The reaction mixture was partitioned (ethyl acetate and water), the aqueous phase was extracted (ethyl acetate), the organic phases were combined, washed (water then brine) then dried (Na₂SO₄) and evaporated. Flash chromatography, eluting with 20% and 60% ethyl acetate/hexane then 100% ethyl acetate, furnished a yellow oil (50 mg, 11%); TLC R_{f}=0.6, ethyl acetate.

### EXAMPLE 9

### 1-(3-(1-Ethoxycarbonyl-1-(5-nitrothien-2-yl)ethoxy)-4-methoxy-phenyl)-2-(3,4,5-trimethoxyphenyl)-Z-ethene

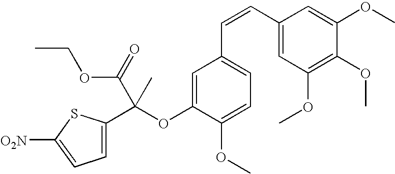

Diisopropyl azodicarboxylate (128 mg, 0.63 mmol) was added dropwise to a solution of ethyl 2-hydroxy-2-(5-nitrothien-2-yl)propanoate (54 mg, 0.22 mmol), combretastatin A4 (100 mg, 0.32 mmol) and triphenylphosphine (166 mg, 0.63 mmol) in THF (1 mL). The reaction mixture was stirred for 16 hours then adsorbed onto flash silica in vacuo. Flash chromatography, eluting with 25% EtOAc/hexane, furnished a mixture of combretastatin A4 and desired product. Further flash chromatography, eluting with 3% EtOAc/DCM, afforded the title compound as a yellow oil (50 mg, 42%). TLC R_{f}=0.2, 30% EtOAc/hexane; LC-RT 5.74 minutes (TFA50-100%); MS m/z 543 (M⁺), 497 (M⁺-NO₂), 316, 301, 283, 252, 241. ¹H NMR (250 MHz, CDCl₃) δ 7.82 (1H, d, J=4.3, HarH), 7.06 (1H, dd, J=8.4, 2.1, ArH), 7.02 (1H, d, J=4.3, HarH), 6.87 (1H, d, J=1.7, A1H), 6.85 (1H, d, J=8.3, ArH), 6.49 (4H, s, CH═CH, 2×ArH), 4.26 (2H, q, J=7.3, CO₂CH₂CH₃), 3.89 (3H, s, OCH₃), 3.85 (3H, s, OCH₃), 3.76 (6H, s, 2×OCH₃), 1.78 (6H, s, 2×CH₃), 1.27 (3H, t, J=7.2, CO₂CH₂CH₃) ppm.

### EXAMPLE 10

### N-(2-{3-[1-Methyl-1-(5-nitro-thiophen-2-yl)-ethoxy]-phenyl}-ethyl)-acetamide

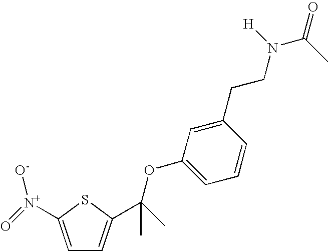

2-(5-nitrothien-2-yl)propan-2-ol (50 mg, 0.27 mmol) was dissolved in benzene (1 ml) together with N-acetyl-3-(2-aminoethyl)phenol (66 mg, 0.44 mmol) under a nitrogen atmosphere. 1,1′-(Azodicarbonyl)dipiperidine (68 mg, 0.27 mmol) was then added, followed by tri-n-butylphosphine (55 mg, 0.27 mmol) in benzene (0.5 ml)) via syringe. The solution was heated under reflux for 7 days, cooled and applied directly to a silica column, which was eluted with ethyl acetate. The product obtained was washed with NaOH (0.1 M) and then re-columned to remove residual N-acetyl-3-(aminoethyl)phenol, to give 43 mg (46%) of the title compound as a yellow waxy solid. MS (m/z, %) 348 (M⁺, 1%), 179 (75%), 170 (100%) LC-RT 5.34 minutes (TFA 50-100%).

1. A compound of formula (1), or a pharmaceutically acceptable salt thereof, 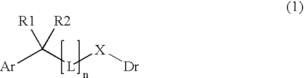
wherein:
Ar is a substituted aryl or heteroaryl group bearing at least one nitro or azido group or is a group of formula (2) or (3) 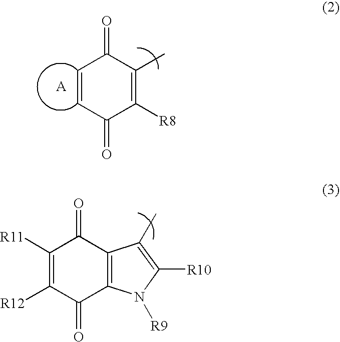
R₁ and R₂, which may be the same or different are independently optionally substituted alkyl, optionally substituted alkenyl, optionally substituted alkynyl, aryl, COR₃ or, together with the intervening carbon atom, form an optionally substituted heterocycloalkyl or carbocyclic ring;
L is —OC(O)— or —OP(O)(OR₆)—;
n is 0 or 1;
X is O, S, NR₇ or a single covalent bond;
R₃ is OR₄ or NR₄R₅;
R₄, R₅, R₆ and R₇ are each independently hydrogen or optionally substituted alkyl or,
where R₃ is NR₄R₅, R₄ and R₅ can be joined to form, together with the intervening nitrogen atom, a heterocycloalkyl ring;
R₈ is hydrogen, alkoxy or dialkylaminoalkyl;
R₉ is optionally substituted alkyl;
R₁₀ is hydrogen, alkyl, alkoxy or dialkylaminoalkyl;
R₁₁ and R₁₂ are independently hydrogen, alkyl, alkoxy, thioalkoxy, amino, alkylamino, dialkylamino, morpholino, piperidino, piperazino or 1-aziridinyl;
A is an optionally substituted aryl or heteroaryl ring; and
Dr is a moiety such that DrXH represents a cytotoxic or cytostatic compound.

2. A compound according to claim 1, wherein the alkyl, alkenyl and alkynyl groups in the R₁ to R₁₂ substituents are unsubstituted or substituted with 1, 2 or 3 unsubstituted substituents selected from halogen, amino, mono(C₁-C₄ alkyl)amino, di(C₁-C₄ alkyl)amino, hydroxy, C₁-C₄ alkoxy, C₁-C₄ alkylthio and (C₁-C₄ alkyl)sulphonyl groups.

3. A compound according to claim 1, wherein aryl and heteroaryl groups in the Ar, A and R₁, R₂ substituents are unsubstituted or substituted with 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy.

4. A compound according to claim 1, wherein the heterocycloalkyl ring and carbocyclic rings in the R₁ to R₃ substituents are unsubstituted or substituted with 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy.

5. A compound according to claim 1, wherein R₁ and R₂, together with the carbon to which they are attached, form a 3 to 10 membered heterocycloalkyl ring or a C₃₋₁₀ carbocyclic ring, which ring is unsubstituted or substituted by 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy.

6. A compound according to claim 5, wherein R₁ and R₂, together with the carbon to which they are attached, form a 5 to 6 membered heterocycloalkyl ring, which ring is unsubstituted or substituted by one unsubstituted C₁-C₂ alkyl group.

7. A compound according to claim 1, wherein R₁ and R₂ are the same or different and each represent unsubstituted C₁-C₆ alkyl, unsubstituted C₁-C₆ alkenyl, unsubstituted C₁-C₆ alkynyl, a COR₃ group, an unsubstituted phenyl group or a phenyl group which is substituted with 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy.

8. A compound according to claim 7, wherein R₁ and R₂ are the same or different and each represent unsubstituted C₁-C₄ alkyl, unsubstituted C₁-C₄ alkenyl, unsubstituted C₁-C₄ alkynyl, a COR₃ group, an unsubstituted phenyl group or a phenyl group which is substituted with 1, 2 or 3 unsubstituted substituents selected from halogen, C₁-C₄ alkyl, hydroxy, amino, C₁-C₂ haloalkyl, C₁-C₂ alkoxy and C₁-C₂ haloalkoxy.

9. A compound according to claim 7, wherein R₃ is hydroxy, unsubstituted C₁-C₄ alkoxy or NR₄R₅, wherein R₄ and R₅ are the same or different and each represent hydroxy or unsubstituted C₁-C₄ alkoxy, or R₄ and R₅ form, together with the nitrogen atom to which they are attached, a 3 to 10 membered heterocycloalkyl ring, which ring is unsubstituted or substituted by 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy.

10. A compound according to claim 9, wherein R₃ is hydroxy, unsubstituted C₁-C₂ alkoxy or NR₄R₅, wherein R₄ and R₅ are the same or different and each represent hydrogen or unsubstituted C₁-C₄ alkyl.

11. A compound according to claim 7, wherein R₁ and R₂ are the same or different and each represent unsubstituted C₁-C₂ alkyl or an unsubstituted —CO₂—(C₁-C₂ alkyl) group.

12. A compound according to claim 1, wherein n is 0 and X is O or S.

13. A compound according to claim 1, wherein n is 1 and X is NH.

14. A compound according to claim 1, wherein n is 1 and L is —OC(O)— or —OP(O)(OR₆), wherein R₆ is hydrogen or unsubstituted C₁₋₆ alkyl.

15. A compound according to claim 14, wherein L is —OC(O)—.

16. A compound according to claim 1, wherein Ar is a substituted aryl or heteroaryl group, which group carries one substituent selected from nitro and azido substituents and 0, 1 or 2 further unsubstituted substituents chosen from halogen, C₁-C₆ alkyl, hydroxy, amino, C₁-C₄ haloalkyl, C₁-C₄ alkoxy and C₁-C₄ haloalkoxy substituents.

17. A compound according to claim 16, wherein Ar is a phenyl group or a 5- or 6-membered heteroaryl group, which group carries only one substituent which substituent is selected from nitro and azido substituents.

18. A compound according to claim 17, wherein Ar is an unsubstituted group selected from nitrophenyl, nitroimidazole, nitrothiophene and nitrofuranyl groups.

19. A compound according to claim 1, wherein DrXH is selected from an anthracyclin antibiotic, an antimetabolite, a topoisomerase inhibitor, an inhibitor of mitosis, inhibitors of protein kinases and an antagonist of (6R)-5,6,7,8-tetrahydrobiopterin.

20. A compound according to claim 19, wherein DrXH is selected from doxorubicin, epirubicin, daunorubicin, 5-fluorouracil, 6-mercaptopurine, 6-thioguanine, cytarabine, gemcitabine, capecitabine, fludarabine, cladribine, decitabine (5-aza-2′-deoxycytidine), troxacitabine (2′-deoxy-3′-oxacytidine), 5-azacytidine, 4′-thioaracytidine, tezacitabine, clofarabine, trimetrexate and methotrexate, etoposide and teniposide, topotecan, SN38, combretastatin A4, combretastatin A1, podophyllotoxin, vinblastine, vincristine vinorelbine, paclitaxel and docetaxel, an epothilone, deoxyepothilone B BMS 247550, a dolastatin derivative, a cryptophycin derivative, gefitinib, erlotinib, ZD6474 and AZD2171.

21. A compound according to claim 20, wherein DrXH is combretastatin A4, etoposide, cytarabine or 6-mercaptopurine.

22. A compound according to claim 1 which is 1-(4-Methoxy-3-(2-(5-nitrothiophen-2-yl) propan-2-yl)oxyphenyl-2-(3,4,5-trimethoxy)phenyl-Z-ethene, 1-(4-Methoxy-3-(2-(4-nitrophenyl)propan-2-yl) oxyphenyl-2-(3,4,5-trimethoxy)phenyl-Z-ethene, 9-(7,8-Dihydroxy-2-methyl-hexahydro-pyrano[3,2-d][1,3]-dioxin-6-yloxy)-5-{3,5-dimethoxy-4-[1-methyl-1-(4-nitrophenyl)-ethoxy]-phenyl}-5,8,8a,9-tetrahydro-5aH-furo[3′,4′:6,7]naphtho[2,3-d][1,3]dioxol-6-one, 6-(2-(4-nitrophenyl)propan-2-ylsulfanyl)-9H-purine, 1-(4-Methoxy-3-(1-methyl-4-(5-nitrothien-2-yl)piperidin-4-yl)oxycarbonyloxy)phenyl -2-(3,4,5-trimethoxy)phenyl-Z-ethene, 1-(4-Methoxy-3-(2-(1-methyl-2-nitroimidazol-5-yl) propan-2-yl)oxyphenyl-2-(3,4,5-trimethoxy)phenyl-Z-ethene, 6-(2-(5-nitrothien-2-yl)propan-2-ylsulfanyl)-9H-purine, N⁴-(2-(5-nitrothien-2-yl) prop-2-yl)oxycarbonyl-1-β-D-arabinofuranosylcytosine, 1-(3-(1-Ethoxycarbonyl-1-(5-nitrothien-2-yl)ethoxy)-4-methoxy-phenyl)-2-(3,4,5-trimethoxyphenyl)-Z-ethene and N-(2-{3-[1-Methyl-1-(5-nitro-thiophen-2-yl)-ethoxy]-phenyl}-ethyl)-acetamide, or a pharmaceutically acceptable salt thereof.

23. A pharmaceutical composition comprising a compound according to claim 1, or a pharmaceutically acceptable salt thereof, and a pharmaceutically acceptable carrier or diluent.

24. A method of ameliorating or reducing the incidence of a proliferative disorder in a patient, which method comprises administering to said patient an effective amount of a compound as defined in claim 1, or a pharmaceutically acceptable salt thereof.

25. (canceled)

26. A method according to claim 24, wherein the proliferative disorder is cancer, rheumatoid arthritis, psoriatic lesions, diabetic retinopathy or wet age-related macular degeneration.

27. A method according to claim 24, wherein the proliferative disorder is a hypoxic disorder.

28. A method according to claim 24, wherein the proliferative disorder is a solid tumour or leukaemia.

29. (canceled)

30. A method according to claim 24, which method comprises administering to said patient an effective amount of
(a) a compound as defined in claim 1, or a pharmaceutically acceptable salt thereof; and
(b) a reductase, an anti-body reductase conjugate, a macromolecule-reductase conjugate or DNA encoding a reductase gene.

31. (canceled)
